(19) United States
(12) Patent Application Publication
(10) Pub. No.: US 2004/0146867 A1
(43) Pub. Date: Jul. 29, 2004

(54) Compounds and processes for single-pot attachment of a label to siRNA

(76) Inventors: Paul M. Slattum, Madison, WI (US); Vladimir G. Budker, Madison, WI (US); James E. Hagstrom, Madison, WI (US); Jon A. Wolff, Madison, WI (US)

Correspondence Address: Mark K. Johnson, Mirus Corporation, 505 S. Rosa Rd, Madison, WI, 53719, US

(21) Appl. No.: 10/350,725

(22) Filed: Jan. 24, 2003

(51) Int. Cl. C12Q 1/68; C07H 21/04; C07H 21/02

(52) U.S. Cl. 435/6; 536/25.32

(57) ABSTRACT

Compounds and methods are provided for a single-pot covalent attachment of a label to an siRNA comprising forming a covalently attachable labeling reagent for alkylating the molecule. Then, combining the covalently attachable labeling reagent with a mixture containing the molecule, under conditions wherein the labeling reagent has reactivity with the molecule thereby forming a covalent bond.

### FIELD

The described invention relates to compounds and methods for covalently attaching a label to an siRNA. More specifically, the compounds are alkylating compounds having a reporter molecule and the covalent attachment is performed in a one-pot alkylation reaction.

### BACKGROUND

Small interfering RNAs mediate a biological phenomenon termed RNA interference (RNAi). RNAi is the process wherein double-stranded RNA (dsRNA), when present in a cell, inhibits expression of a gene that has an identical or nearly identical sequence. Inhibition is caused by degradation of the messenger RNA (mRNA) transcribed from a target gene (Sharp 2001). Biochemical analyses suggest that dsRNA introduced into the cytoplasm of a cell is first processed into RNA fragments 21-25 nucleotides long (Hammond et al 2000; Hamilton and Baulcombe 1999; Zamore et al 2000; Yang et al 2000; Parrish et al 2000). Data obtained from studies in which siRNA, 21-25 base pairs in length, was delivered to mammalian cells in culture indicated that sequence-specific inhibition through RNAi is indeed effective (Caplen et al 2001; Elbashir et al 2001 a). These siRNAs likely act as guides for mRNA cleavage, as the target mRNA is cleaved at a position in the center of the region covered by a particular siRNA (Elbashir et al 200 lb). Evidence suggests that the siRNA is part of a multicomponent nuclease complex termed the RNA-induced silencing complex (RISC) (Hammond et al 2000).

The ability to tag or label siRNA simply and reliably is attractive for a wide variety of molecular and cellular biology applications. Some specific applications in which a labeled siRNA probe can be used include nucleic acid localization studies, quantitation, RNase quantitation, and hybridization reaction procedures.

Both enzyme mediated and direct labeling protocols have been developed to attach detectable tags or markers such as radioactive molecules, fluorescent compounds, biotin, haptens/antigens/epitopes, etc. to DNA and RNA. While these labeling methods have allowed sensitive detection systems there remains significant disadvantages with each of the labeling systems developed to date. Enzymatic labeling systems require a number of reagents including both unlabeled and labeled nucleotide precursors, primers, and/or enzymes to facilitate nucleic acid synthesis. Labeling efficiency is not easily controlled with these systems and the original nucleic acid molecule is not the component that is labeled. Current chemical methods developed for direct labeling of nucleic acids include: introduction of primary amines on cytosine by sodium bisulfite in the presence of a diamine, transamination of cytosine bases by 4-aminohydroxybutylamine (Adarichev et al 1995), modification of the C-8 position of adenine or guanine by diazonium salt and sodium nitrite, modification of guanine with 2-acetylaminofluorene converted to N-acetoxy-2-acetylaminofluorene (Landegent et al 1984), and hydrazine reaction with a ring-opened guanine. In 1967 Belikova et al. (Belikova et al 1967) first described monoadduct alkylation of ribonucleosides and diribonucleoside phosphates using 2-chloroethylamine residues. While this work provided evidence that ribonucleosides could be covalently modified with an alkylating mustard derivative, the efficiency of the process was very low. Utilizing a multi-step process, Frumgarts et al. (Frumgarts et al 1986) alkylated DNA using the nitrogen mustard 4-(N-methylamino-N-2-chloroethyl) benzylamine, and subsequently attached fluorescent labels to the amine that had been covalently attached to the DNA. This multi-step process required that the mustard and fluorescent label be used in a large molar excess to the DNA being labeled. These labeling methods have significant limitation including: laborious multi-step protocols, modification of amines involved in base pairing, derivatization of only single stranded DNA, low efficiency and/or high variability of labeling, and harsh reaction conditions and/or unstable reactants.

There are a wide variety of reporter molecules that may be employed for covalent attachment to a labeling reagent that are useful in detection systems. All that is required is that the reporter molecule can be covalently attached to the labeling reagent and provide a signal that can be detected by appropriate means. Reporter molecules may be radioactive or non-radioactive. Non radioactive reporter molecules include fluorescent compounds, proteins, and affinity molecules (e.g. digoxin, biotin, DNP)

### BRIEF DESCRIPTION OF DRAWINGS

FIGS. 1A-1B. The diagram in (A) illustrates the general structure for an siRNA labeling reagent wherein D is a label or tag, B is a linker that increases affinity of the labeling reagent for nucleic acid; and, A is a mustard or three-membered ring alkylating agent. The structure in (B) illustrates an example of a useful linker segment containing positive charge. The positive charge increases affinity of the labeling reagent for nucleic acid.

FIGS. 2A-2J. Illustrations of the chemical structures for:

A. 3-bromo-1-(trifluoroacetamidyl)propane

B. N,N-dimethyl-N-[N′-(tert-butoxycarbonyl)-3-aminopropylamine]

C. N-[N′-(tert-butoxycarbonyl)-3-aminopropyl]-N,N-dimethyl-3-aminopropylammonium salt

D. N-[N′-{4-[(2-chloroethyl)-methylamino]-benzylamine}-3-aminopropyl]-N,N-dimethyl-3-aminopropylammonium salt

E. Label-IT Cy™3 siRNA labeling reagent

F. Label IT-Cy™5 siRNA labeling reagent

G. Label-IT® Fluorescein

H. Label-IT® Carboxy-X-Rhodamine (CX-RH)

I. Label-IT® Tetramethyl Rhodamine (TM-RH)

J. Label-IT® Biotin

FIG. 3. SiRNA covalently labeled with the compound shown in FIG. 2E allows the visual tracking of siRNA delivered to a cell. SiRNA was labeled with FIG. 2E and delivered to CHO cells with TransIT TKO. Cy3-labeled siRNA is shown in white. Cells were visualized by reflected light and are shown in grey.

FIGS. 4A-4F. The diagram in (A) illustrates the structure of an ineffective labeling reagent bearing a net charge of −1 at pH 7. (B) illustrates the reaction intermediate for (A) showing the positive charge gained. The diagram in (C) illustrates an effective labeling reagent bearing a net neutral charge. (D) illustrates the reaction intermediate for (C) showing the positive charge gained on the aziridine, bringing the net charge of the labeling reagent reactive species to +1. The diagram in (E) illustrates an ineffective labeling reagent bearing a net neutral charge. (F) illustrates the reaction intermediate for (E) showing no positive charge gained, leaving the net charge of the labeling reagent reactive species at neutral.

### SUMMARY

In a preferred embodiment, we describe siRNA labeling reagents that utilize the nucleic acid alkylating ability of mustards and three-membered ring compounds. The components of the labeling reagent consist of a mustard or three-membered ring moiety and a label or tag. The labeling reagent may also contain a linker or spacer group and/or an affinity group. Mustards include nitrogen and sulfur mustard. Three-membered ring compounds include those with nitrogen, sulfur, and oxygen heteroatoms. A reactive nitrogen mustard derivative used in the synthesis of these labeling agents can be the aromatic nitrogen mustard 4-[(2-chloroethyl)-methylamino]-benzaldehyde. This nitrogen mustard derivative was described in U.S. Pat. No. 2,141,090. The label or tag can be a detectable marker or a functional group. The label can be used to detect the siRNA, to attach a functional group to the siRNA, or to covalently or non-covalently crosslink the labeled-siRNA to another compound.

In a preferred embodiment, we describe an siRNA labeling method that combines one-pot simplicity with high efficiency labeling and results in a labeled siRNA that remains intact and stable. The procedure for labeling results in the formation of a covalent bond between the labeling reagent and the siRNA. The labeling procedure comprises: forming a covalently attachable labeling reagent for alkylating the siRNA, combining the labeling reagent with a mixture containing the siRNA under conditions wherein the labeling reagent has reactivity with the siRNA thereby forming a covalent bond, and separation of the labeled siRNA from the unreacted labeling reagent. The extent of labeling can be controlled by regulating the relative amounts of labeling reagent and siRNA, by adjusting the length of the incubation of the labeling reagent with the siRNA, by controlling the temperature of the incubation, by controlling the absolute concentrations of the siRNA and labeling reagent, and by controlling the composition of the aqueous or organic solution in which the labeling reaction occurs.

In a preferred embodiment, we describe compounds, called labeling reagents, for the covalent attachment of a label to siRNA comprising: an alkylating group covalently linked to a label wherein the labeling reagent has affinity for nucleic acid when the bond between the labeling reagent and the siRNA is formed. The alkylating group may be a mustard or a three-membered ring containing group selected from the list comprising: nitrogen mustards, sulfur mustards, aziridines, oxiranes (epoxides), episulfides, and cyclopropanes. A preferred nitrogen mustard is an aromatic mustard. A preferred aromatic mustard is an aromatic tertiary nitrogen mustard. A preferred aromatic tertiary nitrogen mustard is 4-[(2-chloroethyl)-methylamino]-benzaldehyde. The label may be selected from the group comprising: fluorescence-emitting compounds, radioactive compounds, haptens, immunogenic molecules, chemiluminescence-emitting compounds, proteins, and functional groups. Preferred fluorescence-emitting compounds are fluorescent compounds useful for fluorescence miscroscopy and microarray analyses such as fluorescein, rhodamine and cyanine dyes and their derivatives. The labeling reagent may further contain groups that alter the affinity of the reagent for nucleic acid, such as cationic groups, minor groove binding groups and major groove binding groups, groups that alter the solubility of the reagent, or linker/spacer groups that increase the linkage distance between the components of the labeling reagent.

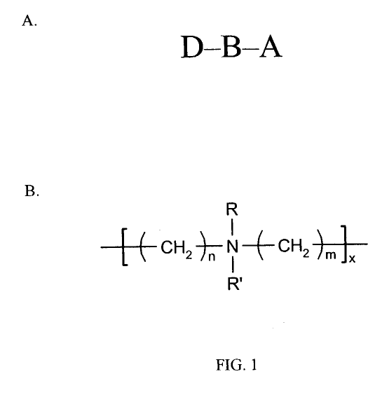

In a preferred embodiment, a compound is provided comprising the general structure shown in FIG. 1A, wherein D is a label selected from the group comprising detectable markers (e.g., fluorescence-emitting compounds, radioactive groups, haptens, affinity groups, immunogenic molecules, chemiluminescence-emitting compounds, proteins) and functional groups; B is a linker and may provide affinity for nucleic acid by interactions comprising electrostatic, minor groove binding, major groove binding, and intercalation; and, A is selected from the group of alkylating agents consisting of mustards and three-membered ring derivatives. B or D may also contain groups that increases the linkage distance between the label or tag and the alkylating agent. An example of such a group is polyethyleneglycol (PEG). A preferred linker segment (B) that provides affinity for nucleic acid comprises the general structure shown in FIG. 1B, wherein, R is selected from the group of alkyls and hydrogen, R′ is selected from the group of alkyls and hydrogen, n is an integer from 1 to 20, m is an integer from 1 to 20, and x is an integer from 1 to 5. The labeling reagent itself may be detectable (e.g., where D is a radioactive group, a fluorescent compound or an enzyme) without further treatment. Alternatively, the labeling reagent may contain a tag that can interact, either covalently or non-covalently, with another compound which can be detected (e.g., where D is an affinity group such a biotin which can interact with a labeled streptavidin or anti-biotin antibody).

Labeling of the siRNA can be used for several purposes including, but not limited to: a) determination of the sub-cellular and tissue localization of siRNA that is delivered to cells in vitro or in vivo; b) quantitation of siRNA; c) covalently attaching functional groups; d) detection of nucleic acids or proteins using techniques that rely upon hybridization or binding affinity of the labeled siRNA to target nucleic acid or protein; and, e) crosslinking the siRNA to another compound.

In a preferred embodiment, a kit is provided comprising: a receptacle containing a covalently attachable labeling reagent for alkylating an siRNA in a single-pot reaction. Instructions for use are also provided with the kit. By the term instructions for use, it is meant a tangible expression describing the reagent concentration for at least one assay method, parameters such as the relative amount of reagent and sample to be admixed, maintenance time periods for reagent/sample admixtures, temperature, buffer conditions and the like.

Reference is now made in detail to the preferred embodiments of the invention, examples of which are illustrated in the accompanying drawings.

### DETAILED DESCRIPTION

Definitions:

1. alkylation—A chemical reaction that results in the attachment of an alkyl group to the substance of interest, a nucleic acid in a preferred embodiment.

2. alkyl group—An alkyl group possesses an sp3 hybridized carbon atom at the point of attachment to a molecule of interest.

3. aqueous or non-aqueous solutions—Aqueous solutions contain water. Non-aqueous solutions are made up of organic solvents

4. aziridine—A three-membered ring containing one nitrogen atom.

5. bifunctional—A molecule with two reactive ends. The reactive ends can be identical as in a homobifunctional molecule, or different as in a heterobifucnctional molecule.

6. buffers—Buffers are made from a weak acid or weak base and their salts. Buffer solutions resist changes in pH when additional acid or base is added to the solution.

7. combinatorial techniques—Techniques used to prepare and to screen extremely large pools of polynucleic acid sequences in which the sequences are in known positions on “chips”, multiwell devices, multislot devices, beads, or other devices capable of segregating the polynucleic acid sequences.

9. crosslinking—The chemical attachment of two or more molecules with a bifunctional reagent.

10. cyclopropane—A three-membered ring made up of all carbon atoms.

11. electrostatic interactions—The non-covalent association of two or more substances due to attractive forces between positive and negative charges.

12. enzyme—Proteins for the specific function of catalyzing chemical reactions.

13. episulfide—A three-membered ring containing one sulfur atom.

14. hapten—A small molecule that cannot alone elicit the production of antibodies to itself.

However, when covalently attached to a larger molecule it can act as an antigenic determinant, and elicit antibody synthesis.

15. hybridization—Highly specific hydrogen bonding system in which guanine and cytosine form a base pair, and adenine and thymine (or uracil) form a base pair.

16. imine—A compound derived from ammonia and containing a bivalent nitrogen combined with a bivalent nonacid group, i.e. the nitrogen atom is linked to a carbon atom by a double bond (C═N—H or C═N—C) In contrast, in a amine, all atoms are covalently attached to the nitrogen via single bonds.

17. intercalating group—A chemical group characterized by planar aromatic ring structures of appropriate size and geometry capable of inserting themselves between base pairs in double-stranded DNA.

18. label—Labels include reporter or marker molecules or tags such as chemical (organic or inorganic) molecules or groups capable of being detected, and in some cases, quantitated in the laboratory. Reporter molecules may be selected from the group comprising: fluorescence-emitting molecules (which include fluoresceins, rhodamines, cyanine dyes, hemi-cyanine dyes, pyrenes, lucifer yellow, BODIPY®, malachite green, coumarins, dansyl derivatives, mansyl derivatives, dabsyl drivatives, NBD flouride, stillbenes, anthrocenes, acridines, rosamines, TNS chloride, ATTO-TAG™, Lissamine™ derivatives, eosins, naphthalene derivatives, ethidium bromide derivatives, thiazole orange derivatives, ethenoadenosines, CyDyes™, aconitine, Oregon Green, Cascade Blue, IR Dyes, Thiazole Orange PMS-127-184, Oregon Green PMS-144-19, BODIPY®-FI PMS-144-20, TAMRA, green fluorescent protein (GFP), and other fluorescent molecules), immunogenic molecules, haptens (such as digoxin), affinity molecules (such as biotin which binds to avidin and streptavidin), chemiluminescence-emitting molecules, phosphorescent molecules, oligosaccharides which bind to lectins, proteins or enzymes (such as luciferase, β-galactosidase and alkaline phosphatase), and radioactive atoms or molecules (such as H³, C¹⁴, P32, P33, S35, I²⁵, I¹³¹, Tc⁹⁹, and other radioactive elements). Labels also include functional groups which alter the behavior or interactions of the compound or complex to which they are attached. Functional groups may be selected from the list comprising: cell targeting signals, nuclear localization signals, compounds that enhance release of contents from endosomes or other intracellular vesicles (releasing signals), peptides (which include nuclear localization signals, polyArginine, polyHistidine, cell permeable peptides, etc.), hydrophobic or alkyl groups (such as dioleoyl and stearyl alkyl chains), and reactive groups (selected from the list comprising: carboxylic acids, amines, bromoacetamides, dibromoacetamides, PDPs, thiols, polyacids, chelators, mustards, disulfides, chelators, peptides, ligands, hydrophobic groups, and PEG).

19. labeling—Attachment of a reporter molecule or tag via a chemical bond to a compound of interest such as a nucleic acid or protein.

20. labeling reagent—A compound containing a reporter molecule, label, or tag that can be covalently attached to a nucleic acid or a protein

21. minor groove binding group—A chemical group with an affinity for the minor groove of double stranded DNA through non-covalent interactions.

22. major groove binding group—A chemical group with an affinity for the major groove of double stranded DNA through non-covalent interactions.

23. Mustards, including nitrogen mustards and sulfur mustards—Mustards are molecules consisting of a nucleophile and a leaving group separated by an ethylene bridge. After internal attack of the nucleophile on the carbon bearing the leaving group, a strained three membered group is formed. This strained ring (in the case of nitrogen mustards an aziridine ring is formed) is very susceptible to nucleophilic attack, thus allowing mustards to alkylate weak nucleophiles such as nucleic acids. Mustards which have one of the ethylene bridged leaving groups attached to the nucleophile are sometimes referred to as half-mustards. Mustard which have two of the ethylene bridged leaving groups attached to the nucleophile can be referred to as bis-mustards. Examples:

a) nitrogen mustard—A molecule that contains a nitrogen atom and a leaving group separated by an ethylene bridge, i.e. R₂NCH₂CH₂X wherein R=any chemical group, and X=a leaving group, typically a halogen.

b) aromatic nitrogen mustard—RR¹NCH₂CH₂X, wherein: R=any chemical group, R¹=an aromatic ring, N=nitrogen, and X=a leaving group, typically a halogen.

c) bis nitrogen mustard—RN(CH₂CH₂X)₂, wherein: R=any chemical group, N=nitrogen, and X=a leaving group, typically a halogen

d) sulfur mustard—RSCH₂CH₂X, wherein: R=any chemical group, S=sulfur, and X a leaving group, typically a halogen

e) aromatic sulfur mustard—RSCH₂CH₂X, wherein: R=an aromatic ring, S=sulfur, and X=a leaving group, typically a halogen

f) bis sulfur ustard —S(CH₂CH₂X)₂, wherein: S=sulfur and X=a leaving group, typically a halogen

g) selenium mustard—A molecule that contains a nitrogen atom and a leaving group separated by an ethylene bridge, i.e. R₂SeCH₂CH₂X wherein R=any chemical group, and X=a leaving group, typically a halogen.

h) aromatic selenium mustard—RR¹SeCH₂CH₂X, wherein: R=any chemical group, R=an aromatic ring, N=nitrogen, and X=a leaving group, typically a halogen.

i) bis selenium mustard—RSe(CH₂CH₂X)₂, wherein: R=any chemical group, N=nitrogen, and X=a leaving group, typically a halogen

24. nucleic acid—Also, polynucleic acid or polynucleotide. Refers to a string of at least two base-sugar-phosphate combinations. Natural nucleic acids have a phosphate backbone, artificial nucleic acids may contain other types of backbones, but contain the same bases. Nucleotides are the monomeric units of nucleic acid polymers. The term includes deoxyribonucleic acid (DNA) and ribonucleic acid (RNA). RNA may be in the form of an tRNA (transfer RNA), siRNA, snRNA (small nuclear RNA), rRNA (ribosomal RNA), mRNA (messenger RNA), anti-sense RNA, and ribosymes. DNA may be in form plasmid DNA, viral DNA, linear DNA, or chromosomal DNA or derivatives of these groups. In addition these forms of DNA and RNA may be single, double, triple, or quadruple stranded. The term also includes PNAs (peptide nucleic acids), phosphothionates, and other variants of the phosphate backbone of native polynucleic acids.

25. oligonucleotide—A polynucleic acid with 50 or fewer base-sugar-phosphate groups.

26. oxirane—A three-membered ring containing one oxygen atom, also called an epoxide.

27. protein—a molecule made up of 2 or more amino acids. The amino acids may be naturally occurring, recombinant or synthetic.

28. Radioactive detectable markers are characterized by one or more radioisotopes of phosphorous, iodine, hydrogen, carbon, cobalt, nickel, and the like. Detection of radioactive reporter molecules is typically accomplished by the stimulation of photon emission from crystalline detectors caused by the radiation, or by the fogging of a photographic emulsion.

29. R-chloride—The aromatic nitrogen mustard 4-[(2-chloroethyl)-methylamino]-benzylamine

30. R-aldehyde—The aromatic nitrogen mustard 4-[(2-chloroethyl)-methylamino]-benzaldehyde

31. salts—Salts are ionic compounds that dissociate into cations and anions when dissolved in solution. Salts increase the ionic strength of a solution, and consequently decrease interactions between polynucleic acids with other cations.

32. single-pot reaction—A reaction set up to take place after all of the reagents necessary to perform covalent attachment are placed in contact with each other in a receptacle, without further steps. Also called a one-pot reaction.

33. siRNA—SiRNA comprises a double stranded nucleic acid structure typically containing 15-50 base pairs and preferably 21-25 base pairs and having a nucleotide sequence identical or nearly identical to an expressed target gene or RNA within the cell.

34. Slot Blots—Technique in which the polynucleic acid is immobilized on a nylon membrane or nitrocellulose filter using a slot blot apparatus before being probed with labeled polynucleic acid.

One can determine whether or not a particular compound is suitable for the present invention by comparing the candidate compound with successful compounds illustrated in the examples. A suitable alkylating compound will alkylate a target molecule in a one-pot reaction. The examples demonstrate suitable methods and preparation of compounds for successful alkylation of siRNA. A compound suitable for use with the present invention minimally consists of an alkylating group and a label (components A and D below). Suitable compounds may also contain a spacer group (component S below) or an additional component to increase affinity of the labeling reagent for nucleic acid or alter the charge of the labeling reagent (component B below):

A—Alkylating group—chemical functionalities that are electrophilic, allowing them to become covalently attached to compounds bearing a nucleophilic group. Alkylating reagents include mustards (nitrogen mustards and sulfur mustards); and three-membered rings (aziridines, oxiranes, cyclopranes, activated cyclopropanes, and episulfides), including charged three-membered rings.

D—Label: reporter group (detectable marker) or functional group.

reporter group—a chemical moiety attached to the compound for purposes of detection. The reporter molecule may be fluorescent, such as a rhodamine or flourescein derivative or a cyanine dye. The reporter molecule may be a hapten, such as digoxin, or a molecule which binds to another molecule such as biotin which binds to avidin and streptavidin or oligosaccharides which bind to lectins. The reporter molecule may be a protein or an enzyme such as alkaline phosphatase. The reporter molecule may also be or contain radioactive atoms such as H³, C¹⁴, P³², P³³,S³⁵, I¹²⁵, I¹³¹,Tc⁹⁹, and other radioactive elements.

functional group—a group that adds functionality. This group comprises: reactive groups, charged groups, alkyl groups, polyethyleneglycol, ligands, and peptides. A reactive group is capable of undergoing further chemical reactions. Reactive groups include, but are not limited to: alkylating groups (including mustards and three-membered rings), amines, alcohols, thiols, isothiocyanates, isocyanates, acyl azides, N-hydroxysuccinimides, sufonyl chlorides, aldehydes, epoxides, carbonates, imidoesters, carboxylates, alkylposphates, arylhalides (such as difluoro-dinitrobenzene), iodoacetamides, maleimides, aziridines, acryloyl chlorides, flourobenzes, disulfides, succinamides, carboxylic acids, and activated carboxylic groups.

S—Linker/Spacer—a connection, typically between the alkylating group and the label, selected from the group comprising: alkanes, alkenes, esters, ethers, glycerol, amide, saccharides, polysaccharides, heteroatoms such as oxygen, sulfur, or nitrogen, and molecules that are cleavable under physiologic conditions such as a disulfide bridges or enzyme-sensitive groups. The spacer may bear a net positive charge, or be any of the following: minor groove binders, major groove binders, intercalating groups, or other proteins or groups that increase the affinity of the compound for siRNA. The spacer may alleviate possible molecular interference by separating the reporter molecule from the alkylating compound or siRNA after alkylation. The spacer may also contain a group that increases the linkage distance between the label or tag and the alkylating agent (A). The spacer may also increase the aqueous solubility of the labeling reagent.

B—Affinity group—a group that increases affinity of the reagent for nucleic acid or alters the overall charge of the labeling reagent. The affinity group can be attached to the alkylating group, to the label or to the linker/spacer. Alternatively, the affinity group may be incorporated into the linker/spacer. The affinity group may bear a net positive charge or be any of the following: minor groove binders, major groove binders, intercalating groups, or other proteins or groups that increase the affinity of the compound for siRNA. If the other components of the labeling reagent combine to bear a net positive charge, the affinity group may bear a net negative charge, provided the net charge of the reactive species of the labeling reagent is greater than zero. The affinity group may also increase the aqueous solubility of the labeling reagent.

In order for a labeling reagent to be effective, we have found that it is important that the compound have affinity for nucleic acid when the labeling occurs. In other words, the reactive species must have affinity for nucleic acid. This feature serves to increase the affinity of the reagent for the nucleic acid being modified, allowing a functional amount of labeling to occur.

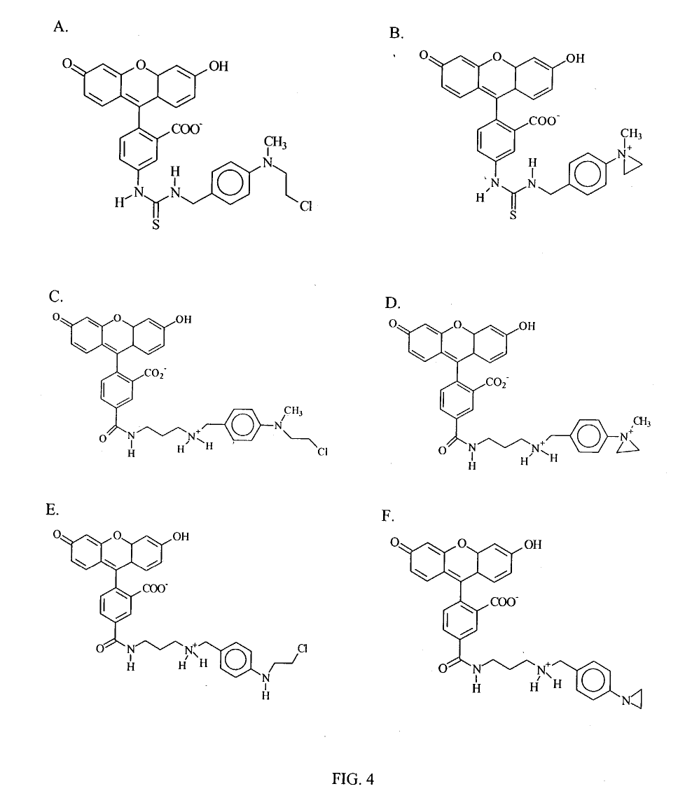

For example, a net charge greater than zero on the labeling reagent when the labeling occurs can provide affinity for nucleic acid. In this example, if the label group carries negative charge, then the linker, alkylating group and affinity group must bear enough combined net positive charge such that the net charge of the reactive species is greater than zero. Thus, the net charge on a labeling reagent can be equal to or greater than zero. A net neutral labeling reagent (charge equal to zero) is effective if the reagent becomes positively charged during the alkylation reaction. As an example, for the compound shown in FIG. 4C, the aromatic nitrogen mustard forms a positively charged aziridine intermediate (FIG. 4D) during the alkylation reaction. FIG. 4C is therefore an effective labeling reagent. If the nitrogen mustard contained a secondary amine (as in FIG. 4E), the intermediate (FIG. 4F) would not gain a positive charge. Thus, for a secondary amine-containing nitrogen mustard, where affinity for nucleic acid is based on charge, the net positive charge on the labeling reagent would need to be greater than zero.

Any of a large number of nucleic acid sequences may be employed in accord with this invention. Included, for example, are target sequences in both RNA and DNA, as are the polynucleotide sequences that characterize various viral, viroid, fungal, parasitic or bacterial infections, genetic disorders or other sequences in target molecules that are desirable to detect. Probes may be of synthetic, semi-synthetic or natural origin.

### EXAMPLES

### Example 1

Synthesis of Labeling Reagents. The synthetic methodology used to prepare the labeling reagents of the invention is described below and in U.S. Pat. No. 6,262,252 incorporated herein by reference.

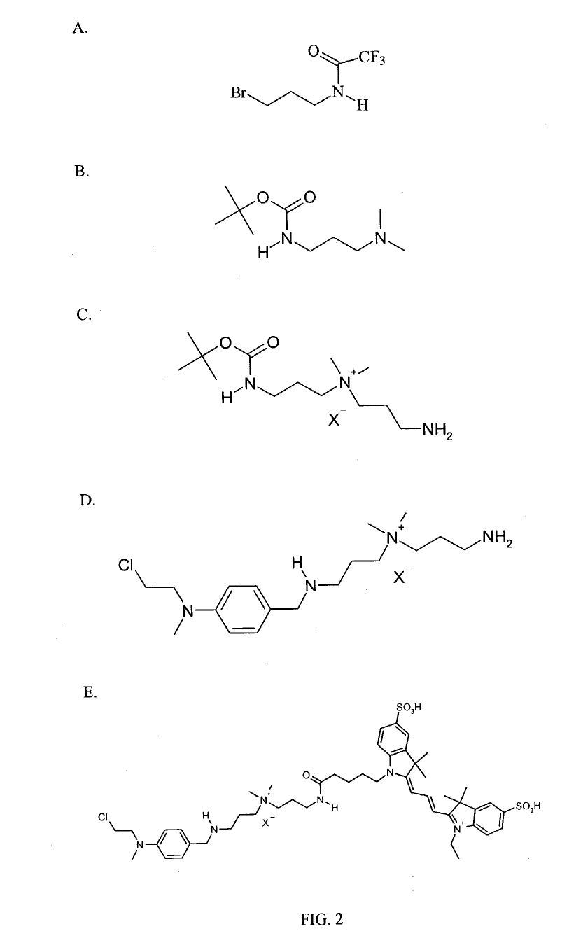

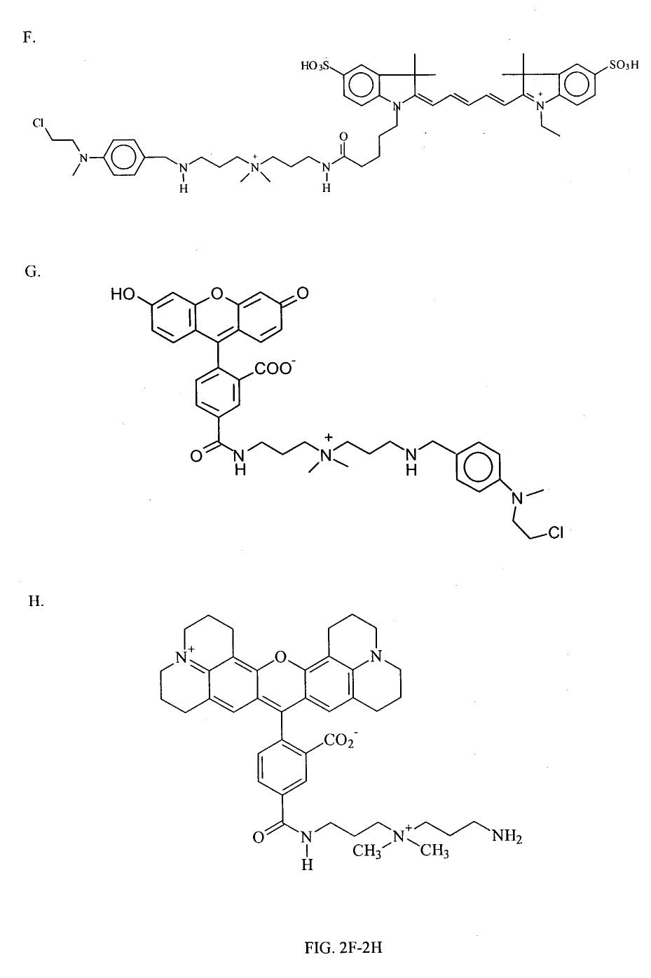

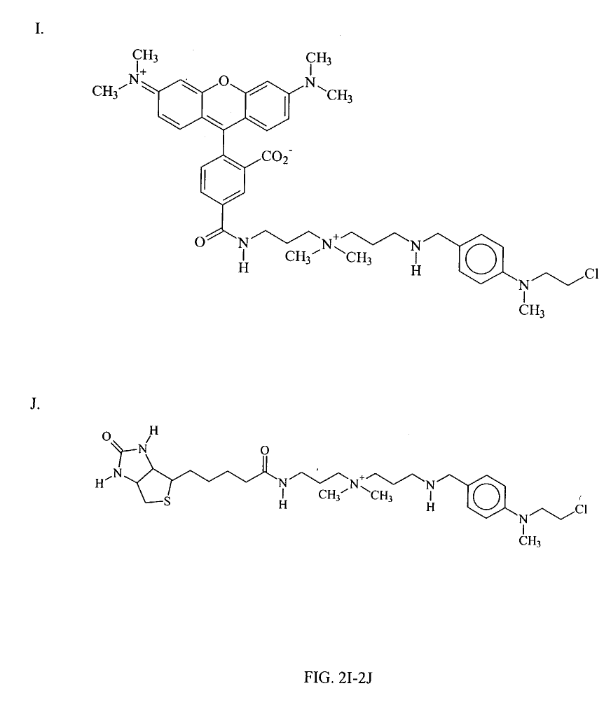

FIG. 2A: Preparation of 3-bromo-1-(trifluoroacetamidyl)propane. To a solution of 3-bromopropylamine (2.19 g, 10.0 mmol, Aldrich Chemical Co., Milwaukee, Wis.) and triethyl amine (1.67 mL, 12.0 mmol, Aldrich Chemical Co.) in 60 mL methylene chloride at 0° C. in a 200 mL roundbottom flask equipped with a addition funnel was added trifluoroacetic anhydride (1.69 mL, 12.0 mmol, Aldrich Chemical Co.) in 60 mL methylene chloride over a period of 20 minutes. The reaction was stirred overnight, washed 1×10 mL 2% bicarbonate, 1×10 mL water, and dried over magnesium sulfate. Removal of solvent yielded 2.07 g (88.5%) product as amorphous crystals. H¹-NMR (CDCl₃): ? 3.55 (m, 2H), 3.45 (m, 2H), 2.17 (m, 2H).

FIG. 2B: N,N-dimethyl-N-[N′-(tert-butoxycarbonyl)-3-aminopropylamine]. 3-dimethylaminopropylamine (251 μL, 204 mg, 2.00 mmol, Aldrich Chemical Co.) was combined with diisopropylamine (348 μL, 2.00 mmol, Aldrich Chemical Co.) in 2 mL tetrahydrofuran. BOC-ON (542 mg, 2.20 mmol, Aldrich Chemical Co.) was added to the stirring reaction mixture. The reaction mixture was stirred at room temperature for 12 hours. Following removal of THF on a rotary evaporator the residue was dissolved in 30 mL diethyl ether, washed 3×2 N NaOH, and dried over MgSO₄. Solvent removal yielded 359 mg (88.7%) product as a colorless oil. H¹⁻-NMR (CDCl₃): δ 5.16 (bs, 1H), 3.76 (m, 2H), 2.30 (m, 2H), 2.21 (s, 6H), 1.65 (m, 2H), 1.44 (s, 9H).

FIG. 2C: N-[N′-(tert-butoxycarbonyl)-3-aminopropyl]-N,N-dimethyl-3-aminopropylammonium carbonate. FIG. 2B (344 mg, 1.70 mmol) and FIG. 2A (433 mg, 1.85 mmol) were combined in 250 μL anhydrous dimethylformamide (DMF), and incubated at 55° C. for 48 hours. Product was precipitated from the reaction mixture by the addition of diethyl ether. Product was dried under vacuum yielding 686 mg (92.5%) product as a colorless oil. H¹-NMR (D₂O): δ 7.95 (s, 1H), 3.45 (m, 2H), 3.35 (m, 4H), 3.20 (m, 2H), 3.10 (s, 6H), 2.10 (m, 2H), 1.95 (m, 2H), 1.45 (s, 9H). The triflouroacetamide group was cleaved by dissolving the reaction product (179 mg, 0.409 mmol) in 1.0 mL methanol and 0.5 mL water. Sodium carbonate (173 mg, 4.09 mmol) was added and the reaction was stirred at room temperature for 12 hours. The carbonate was removed by centrifugation. Product was dissolved in methanol and precipitated by the addition of diethyl ether yielding 93.5 mg (66.5%) product as a colorless solid. TLC: silica gel; water/acetic acid/ethyl acetate; 2/2/1; Rf=0.61, developed using Dragendorffs Reagent. H¹-NMR (CD₃OD): δ 3.37 (m, 4H), 3.15 (m, 8H), 2.73 (m, 2H), 1.94 (m, 4H), 1.44 (s, 9H).

FIG. 2D: N-[N′-{4-[(2-chloroethyl)-methylamino]-benzylamine}-3-aminopropyl]-N,N-dimethyl-3-aminopropylammonium tetra-trifluoroacetate salt. FIG. 2C (123 mg, 0.382 mmol) and 4-[(2-chloroethyl)-methylamino]-benzaldehyde (75.5 mg, 0.382 mmol, kindly provided by V. V. Vlassov, Institute of Bioorganic Chemistry, Siberian Division of the Russian Academy of Sciences, Novosibirsk) were dissolved in 9 mL methanol. Sodium cyanoborohydride (24.0 mg, 0.381 mmol, Aldrich Chemical Co.) was added. The reaction was stirred at room temperature for 18 hours. Solvent was removed from the reaction mixture, the residue was dissolved in TFA, and incubated for 20 minutes at room temperature to remove the BOC protecting group. The TFA was evaporated under a stream of nitrogen, and the residue was purified via HPLC (C-18: acetonitrile/0.1% TFA) to yield 85.0 (27.9%) as a yellow oil. TLC: silica gel; dimethylformamide/acetic acid/water; 1/2/2; Rf=0.31.

FIG. 2E: Label-IT® Cy™3 (Mirus Corporation, Madison, Wis.).

FIG. 2D (100 mg, 0.125 mmol) and Cy3 mono NHS ester (100 mg, 0.130 mmol, Amersham Biosciences) were dissolved in 1.0 mL DMF. Diisopropylethylamine (64.5 mg, 0.5 mmol) was added, and the reaction was stirred at room temperature for 2 hours. The product was purified by HPLC using: column (Aquasil C-18, 250×20 mm, Keystone Scientific), and mobile phase (methanol containing 0.1% trifluoroacetic acid:0.1% trifluoracetic acid, 15 mL/min). Final product was identified by mass spectrometry (PE Sciex 150EX, Perkin-Elmer Biosciences) molecular ion (M⁺, 953 amu).

Many different labeling reagents can be synthesized in a similar manner, by attaching a desired label or tag to the spacer of compound FIG. 2D. Examples include the labeling reagents shown in FIGS. 2F-FJ: F. Label IT-Cy™5, Label-IT® Fluorescein, Label-IT® Tetramethyl Rhodamine, Label-IT® Carboxy-X-Rhodamine and Label-IT® Biotin

### Example 2

Labeling reagents can be covalently attached to siRNA. SiRNA oligomers with overhanging 3′ deoxynucleotides were prepared and purified by PAGE (Dharmacon, LaFayette, Colo.). The luciferase sense oligonucleotide had the sequence: 5′-rCrUrUrArCrGrCrUrGrArGrUrArCrUrUrCrGrATT-3′ (SEQ ID 1), corresponding to positions 155-173 of the reading frame. The luciferase antisense oligonucleotide had the sequence: 5′-rUrCrGrArArGrUrArCrUrCrArGrCrGrUrArArGTT-3′ (SEQ ID 2) corresponding to positionsl73-155 of the reading frame in the antisense direction. The SEAP sense oligomer had the sequence: 5′-rArGrGrGrCrArArCrUrUrCrCrArGrArCrCrArUTT-3′ (SEQ ID 3), corresponding to positions 362-380 of the reading frame. The SEAP antisense oligomer had the sequence: 5′-rArUrGrGrUrCrUrGrGrArArGrUrUrGrCrCrCrUTT-3′ (SEQ ID 4), corresponding to positions 362-380 of the SEAP reading frame in the antisense direction. The letter “r” preceding a nucleotide indicates that the nucleotide is a ribonucleotide. Complementary oligonucleotides were annealed in 100 mM NaCl/50 mM Tris-HCl, pH 8.0 buffer by heating to 94° C. for 2 min, cooling to 90° C. for 1 min, then cooling to 20° C. at a rate of 1° C. per minute. The annealed oligonucleotides containing luciferase and SEAP coding sequence are referred to as siRNA-GL3 and siRNA-SEAP, respectively. The siRNAs were stored at −20° C. prior to use.

Oligomers, siRNA or single stranded oligomer (ssRNA), were labeled with Label-IT® Cy™3, Label-IT® Cy™5, Label-IT® Fluorescein, Label-IT® Tetramethyl Rhodamine (TM-RH), Label-IT® Carboxy-X-Rhodamine (CX-RH), or Label-IT® Biotin labeling reagents (FIGS. 2E-2J; Mirus Corporation, Madison, Wis.) using the following conditions:

TABLE 1
Attachment fluorescent molecules and affinity
molecules to siRNA using
described labeling reagents.

| | | | | | Labeling |
|---|---|---|---|---|---|
| | | | | Abs. | efficiency |
| | Labeling | siRNA | ssRNA | Max. | (bases/ |
| Labeling Reagent | Reagent (μg) | (μg) | (μg) | (nm) | dye) |
| Label-IT ® Cy ™ 3 | 4 | 10 | | 550 | 89.8 |
| | 8 | 10 | | 550 | 57.6 |
| | 6 | | 15^{a} | 550 | 222.3 |
| | 24 | | 15^{a} | 550 | 63.8 |
| Label-IT ® Cy ™ 5 | 4 | 10 | | 649 | 174.4 |
| | 8 | 10 | | 649 | 102.2 |
| | 6 | | 15^{b} | 649 | 379.5 |
| | 24 | | 15^{b} | 649 | 123.1 |
| Label-IT ® FL | 6 | 10 | | 492 | 102.8 |
| Label-IT ® CX-RH | 2 | 10 | | 576 | 815.8 |
| | 3 | 10 | | 576 | 418.4 |
| Label-IT ® TM-RH | 4 | 10 | | 546 | 275.0 |
| | 8 | 10 | | 546 | 162.4 |
| Label-IT ® Biotin | 2 | 10 | | — | — |

^{a}sense strand oligomer
^{b}antisense strand oligomer

Each reaction was cleaned from light in 75 μl 20 mM MOPS pH 7.5 at 37° C. for 1 h. Reactions were then ethanol precipitated, washed in 70% ethanol, and resuspended in 10 μl 100 mM NaCl/50 mM Tris, pH 8.0. The labeling ratio is the μg of labeling reagent relative to the μg of nucleic acid (siRNA). Efficiency of labeling was determined by measuring sample absorption at the appropriate wavelength for each dye (see table) and at 260 nm and 280 nm to quantitate nucleic acid. Cy3-labeled sense strand oligomer was annealed to either Cy5-labeled or unlabeled antisense strand oligomer as above. Similarly, Cy5-labeled antisense strand oligomer was annealed to either Cy3-labeled or unlabeled sense strand oligomer.

The quantitation results demonstrate that the described labeling reagents efficiently label siRNA.

### Example 3

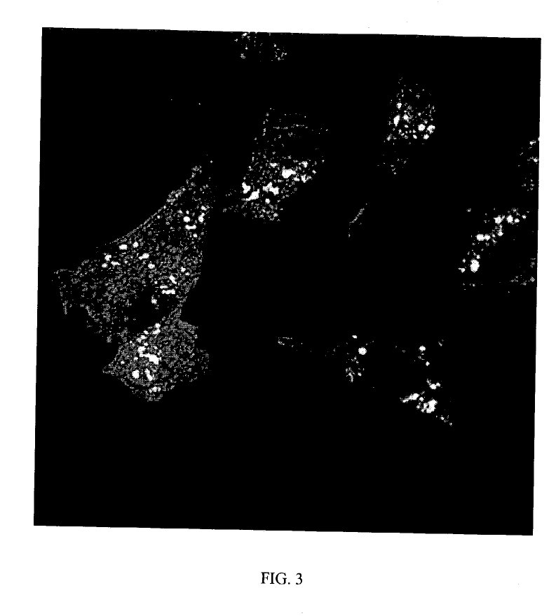

Labeling reagents can be covalently attached to siRNA for use in siRNA localization following cellular delivery. Labeled siRNA-GL3 was transfected into CHO, HeLa or 3T3 cells using TransIT TKO® according to the manufacturer's recommendations. 24 h after transfection, cells were fixed for fluorescence microscopy. Fluorescence was detected using a Zeiss LSM 510 confocal microscope. Strong fluorescent signal with low background was observed (FIG. 3). Labeled siRNA was observed in a punctate pattern accumulating in the perinuclear region; a localization consistent with endocytic internalization of the siRNA. Cells are visible as reflected light at a different wavelength. No fluorescence was observed in cells transfected with unlabeled siRNA.

### Example 4

SiRNA with covalently attached label retains RNAi activity. CHO-LUC and CHO-SEAP cells, carrying a stably integrated and constitutively expressing luciferase or SEAP gene, were maintained in F-12 medium supplemented with 10% fetal bovine serum and G418. All cultures were maintained in a humidified atmosphere containing 5% CO₂ at 37° C. CHO-LUC and CHO-SEAP cells were made by co-transfecting CHO cells (ATCC) in 6-well paltes with a 20 ng of a neomycin resistance gene containing plasmid and 2000 ng of either pCI-Luc or pMIR85, respectively, using TransIT LT1® (Mirus Corporation, Madison, Wis.). Transfected cells were selected by growth in 0.5 mg/ml G418 sulfate. Plasmid pCI-Luc contains the phoitnus pyralis luciferase coding region (pCI-Luc, Promega Corp., Madison, Wis.). Plasmid pMIR85 is similar to pCI-Luc except that the luciferase coding region is replaced by the SEAP coding region.

Approximately 24 h prior to transfection, CHO-LUC and CHO-SEAP cells were plated at an appropriate density in a 24-well plate with 250 μl F12+10% serum. Cells were then transfected with unlabeled, Label-IT labeled or end labeled siRNA-GL3 or siRNA-SEAP using TransIT TKO® (Mirus Corporation, Madison, Wis.) according to the manufacturer's recommendations. SiRNA was labeled as above. 5 nM siRNA-GL3 was used for all CHO-LUC experiments.

Cells were harvested after 24 h or 48 h and assayed for luciferase or SEAP expression. Luciferase activity was measured using the Promega Luciferase Kit (Promega, Madison, Wis.) and a Lumat LB 9507 (EG&G Berthold, Bad-Wildbad, Germany) luminometer. Luciferase activity was recorded in relative light units (RLUs). SEAP expression was measure by a chemiluminescence assay using the Tropix Phospha-Light kit (Applied Biosystems, Forest City, Calif.). Percent inhibition values were adjusted to control cells treated with TransIT TKO without siRNA.

TABLE 2
SiRNA-GL3 covalently modified with a nucleic acid-alkylating
labeling reagent retains RNAi activity and inhibits luciferase
expression when delivered to CHO-LUC cells.

| | | Luciferase | |
|---|---|---|---|
| siRNA | labeling ratio | RLUs | % inhibition |
| TKO control | | 1208800 | |
| GL2 control | | 1123062 | 7.1 |
| GL3 | | 209086 | 82.7 |
| Cy3-GL3 | 0.4:1 | 291099 | 75.9 |
| Cy3-GL3^{a} | 1.6:1 | 185262 | 84.7 |
| Cy5-GL3 | 0.4:1 | 232489 | 80.8 |
| Cy5-GL3^{b} | 1.6:1 | 204718 | 83.1 |
| Cy3/Cy5-GL3^{c} | 0.4:1 | 198329 | 83.6 |
| Cy3/Cy5-GL3^{d} | 1.6:1 | 195447 | 83.8 |
| FL-GL3 | 0.6:1 | 205738 | 83.0 |
| CX-RH-GL3 | 0.2:1 | 243132 | 79.9 |
| TM-RH-GL3 | 0.4:1 | 198532 | 83.6 |
| Biotin-GL3 | 0.2:1 | 203836 | 83.1 |

^{a}Cy3-labeled sense strand, 1.4:1 labeling ratio
^{b}Cy5-labeled antisense strand, 1.4:1 labeling ratio
^{c}Cy3-labeled sense strand, Cy5-labeled antisense strand, 0.4:1 labeling ratio
^{d}Cy3-labelcd sense strand, Cy5-labeled antisense strand, 1.6:1 labeling ratio

TABLE 3
SiRNA-SEAP covalently modified with a nucleic acid-alkylating
labeling reagent retains RNAi activity and inhibits SEAP
expression when delivered to CHO-SEAP cells.

| siRNA | [conc.] | ng/ml SEAP | % inhibition |
|---|---|---|---|
| | | 24h | |
| TKO control | | 2.81 | |
| GL3 control | 25 nM | 3.42 | −23.4 |
| Seap-362 | 1 nM | 0.52 | 83.6 |
| Seap-362 | 3 nM | 0.28 | 91.6 |
| Seap-362 | 25 nM | 0.15 | 95.8 |
| Cy3-Seap-362 | 1 nM | 0.65 | 79.0 |
| Cy3-Seap-362 | 3 nM | 0.42 | 86.8 |
| Cy3-Seap-362 | 25 nM | 0.31 | 90.7 |
| | | 48h | |
| TKO control | | 2.78 | |
| GL3 control | 25 nM | 4.33 | −61.0 |
| Seap-362 | 1 nM | 0.81 | 73.4 |
| Seap-362 | 3 nM | 0.32 | 90.2 |
| Seap-362 | 25 nM | 0.09 | 97.5 |
| Cy3-Seap-362 | 1 nM | 0.56 | 81.9 |
| Cy3-Seap-362 | 3 nM | 0.41 | 87.1 |
| Cy3-Seap-362 | 25 nM | 0.14 | 96.0 |

The labeling reagent FIG. 2E effectively labeled siRNA without affecting RNAi activity, thereby allowing tracking of delivered functional siRNA.

### Example 5

The labeling reagent must have affinity for nucleic acid when the labeling reaction occurs. The aromatic nitrogen mustard fluorescein reagent shown if FIG. 4A has a net charge of −1. During the alkylation reaction, a positively charged aziridine forms bringing the net charge of the labeling reagent intermediate to zero FIG. 4B. The FIG. 4A reagent was found to be unable to label nucleic acids. The reactive species does not have a charge greater than zero. In contrast, the aromatic nitrogen mustard fluorescein labeling reagent shown in FIG. 4C, has a net charge of zero. During the alkylation reaction a positively charged aziridine forms bringing the net charge this labeling reagent intermediate to +1, FIG. 4D. The FIG. 4C labeling reagent was found to efficiently label nucleic acids. The reagent shown in FIG. 4E has a neutral charge, but the nitrogen mustard for this reagent does not form a positively charged intermediate, FIG. 4F. This reagent is not predicted to efficiently label nucleic acid.

The foregoing is considered as illustrative only of the principles of the invention. Further, since numerous modifications and changes will readily occur to those skilled in the art, it is not desired to limit the invention to the exact construction and operation shown and described. Therefore, all suitable modifications and equivalents fall within the scope of the invention.

### REFERENCES

Adarichev V A, Vorobeva N V, Grafodatskii A S, Dymshits G M, Sablina O V. DNA Modification with 4-Aminohydroxybutylamine to Obtain Highly Sensitive Hydridization Probes. Location of Human Choirionic Somatomammotropin Gene. Molecular Biology 1995 293: 538-545,

Belikova A M, Zarytova V F and Grineva N I. Tetrahedron Letters 1967 37:3557-3562. Frumgarts, L. A.; Kipriyanov, S. M.; Kalachikov, S. M.; Dudareva, N. A.; Dymshits, G. M.; Karpova, G. G.; and Salganik, R. I. 1986 Bioorg. Khim. 1211: 1508-1513.

Caplen N J, Parrish S, Imani F, Fire A, Morgan R A. Specific inhibition of gene expression by small double-stranded RNAs in invertebrate and vertebrate systems. Proc Natl Acad Sci USA 2001 98:9742-9747.

Elbashir S M, Harborth J, Lendeckel W, Yalcin A, Weber K, Tuschl T. Duplexes of 21-nucleotide RNAs mediate RNA interference in cultured mammalian cells. Nature 2001 a 411:494-498.

Elbashir S M, Lendeckel W, Tuschl T. RNA interference is mediated by 21- and 22-nucleotide RNAs. Genes Dev 2001b 15:188-200.

Frumgarts L A, Kipriianov S M, Kalachikov S M, Dudareva N A, Dymshits G M. Preparation of fluorescent-labeled DNA and its use as a probe in molecular hybridization. Bioorg Khim. 1986 12(11):1508-1513.

Hamilton A J, Baulcombe D C. A species of small antisense RNA in posttranscriptional gene silencing in plants. Science 1999 286:950-952.

Hammond S M, Bernstein E, Beach D, Hannon G J. An RNA-directed nuclease mediates post-transcriptional gene silencing in Drosophila cells. Nature 2000 404:293-296.

Landegent J E, Jasen in de Wal N, Baan R A, Hoeijmakers J H, Van der Ploeg M. 2-Acetylaminofluorene-modified probes for the indirect hybridocytochemical detection of specific nucleic acid sequences. Exp Cell Res. 1984 1531:61-72.

Parrish S, Fleenor J, Xu S, Mello C, Fire A. Functional anatomy of a dsRNA trigger: differential requirement for the two trigger strands in RNA interference. Mol Cell 2000 6:1077-1087.

Sharp PA. RNA interference-2001. Genes Dev 2001 15:485-490.

Yang D, Lu H, Erickson J W. Evidence that processed small dsRNAs may mediate sequence-specific mRNA degradation during RNAi in Drosophila embryos. Curr Biol 2000 10:1191-1200.

Zamore P D, Tuschl T, Sharp P A, Bartel D P. RNAi: double-stranded RNA directs the ATP-dependent cleavage of mRNA at 21 to 23 nucleotide intervals. Cell 2000 101:25-33.

We claim:

1. A method for single-pot, sequence non-specific, covalent attachment of a label to an siRNA comprising:
a) forming a covalently attachable labeling reagent selected from the group consisting of mustards and three-membered rings for alkylating the siRNA;
b) combining the covalently attachable labeling reagent with a mixture containing the siRNA, under conditions wherein the labeling reagent has sequence non-specific reactivity with the siRNA, thereby forming a covalent bond within an hour.

2. The method of claim 1 wherein the covalently attachable labeling reagent comprises an alkylating compound having a reporter molecule.

3. The method of claim 2 wherein the reporter molecule is selected from the group comprising: fluorescence-emitting molecules, hapten-containing molecules, proteins, radioactive chemicals, and other detectable groups.

4. The method of claim 1 wherein the covalently attachable labeling reagent comprises an alkylating compound having one or more additional functional groups.

5. The method of claim 1 wherein the mustard is selected from the group consisting of nitrogen mustards and sulfur mustards.

6. The method of claim 5 wherein the nitrogen mustard is selected from the group consisting of aromatic nitrogen mustards.

7. The method of claim 1 wherein the label is attachable to the alkylating compound with a spacer.

8. The method of claim 7 wherein the spacer has affinity for nucleic acid.

9. The method of claim 8 wherein the spacer is cationic.

10. An siRNA labeling compound for covalently attaching a label to siRNA comprising: an alkylating group selected from the group consisting of mustards, such as nitrogen mustards and sulfur mustards, and three-membered ring containing compounds, such as aziridines, epoxides, episulfides, and cyclopropanes, covalently linked to one or more labels selected from the group consisting of fluorescence-emitting compounds, radioactive compounds, haptens, immunogenic molecules, chemiluminescence-emitting compounds, proteins, and functional groups; wherein the reactive species has a net charge greater than zero.

11. The labeling compound of claim 10 wherein the label is linked to the alkylating group via a spacer.

12. The labeling compound of claim 11 wherein the spacer is cationic.

13. The labeling compound of claim 10 wherein the alkylating group is an aromatic tertiary-amine containing mustard.

14. An siRNA labeling compound having the structure comprising: 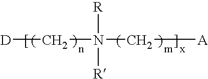
wherein,
D is selected from the group consisting of fluorescence-emitting compounds, radioactive compounds, haptens, immunogenic molecules, chemiluminescence-emitting compounds, proteins, and functional groups;
R is selected from the group of alkyls and hydrogen;
R′ may or may not be present and if present is selected from the group of alkyls and hydrogen;
n is an integer from 1 to 20;
m is an integer from 1 to 20;
x is an integer from 1 to 5; and,
A is selected from the group of alkylating agents consisting of mustards, such as nitrogen mustards and sulfur mustards, and three-membered ring containing compounds, such as aziridines, epoxides, episulfides, and cyclopropanes.

15. A kit comprising:
a) a receptacle containing a covalently attachable labeling reagent for alkylating siRNA in a single-pot reaction; and,
b) instructions for use.


SEQUENCE LISTING (4 sequences)

SEQ ID NO: 1 - DNA, 21 bp, Photinus pyralis
ucgaaguacucagcguaagtt

SEQ ID NO: 2 - DNA, 21 bp, Photinus pyralis
agggcaacuuccagaccautt

SEQ ID NO: 3 - DNA, 21 bp, Homo sapiens
agggcaacuuccagaccautt

SEQ ID NO: 4 - DNA, 21 bp, Homo sapiens
auggucuggaaguugcccutt